(19) United States
(12) Patent Application Publication
(10) Pub. No.: US 2003/0215861 A1
(43) Pub. Date: Nov. 20, 2003

(54) Methods of treating a BRCA associated disorder

(75) Inventors: Grant Alan McArthur, East Melbourne (AU); Andrew John Deans, East Melbourne (AU)

Correspondence Address: SUGHRUE MION, PLLC, 2100 Pennsylvania Avenue, NW, Washington, DC, 20037-3213, US

(73) Assignee: THE PETER MACCULLUM CANCER INSTITUTE

(21) Appl. No.: 10/410,207

(22) Filed: Apr. 10, 2003

(30) Foreign Application Priority Data
Apr. 10, 2002 (AU) PS1659

(51) Int. Cl. A61K 31/00; C12Q 1/68; A01K 67/00

(52) U.S. Cl. 435/6; 800/10; 514/1

(57) ABSTRACT

The present invention relates to methods of treating a BRCA associated disorder, particularly a BRCA1 associated cancer. The invention also provides a method of diagnosis or prognosis of the disorder, preferably a BRCA-1 associated cancer and methods of determining suitable treatment regimes for the disorder or cancer. The invention also provides a transgenic animal useful as a model for testing potential therapeutics for the disorder or cancer.

In a first aspect of the present Invention there is provided a method of treating a BRCA associated disorder, said method comprising modulating Interaction between a BRCA gene or gene product and a p27 gene or gene product.

The present invention relates to methods of treating a BRCA associated disorder, particularly a BRCA1 associated cancer. The invention also provides a method of diagnosis or prognosis of the disorder, preferably a BRCA-1 associated cancer and methods of determining suitable treatment regimes for the disorder or cancer. The invention also provides a transgenic animal useful as a model for testing potential therapeutics for the disorder or cancer.

### BACKGROUND

Proteins encoded by the breast cancer susceptibility genes (BRCA proteins) have been Implicated in a predisposition to breast, ovarian and other cancers. These proteins are ubiquitously expressed thereby implicating them in many processes fundamental to all cells including DNA repair and recombination, checkpoint control of cell cycle and transcription.

Specifically, genetic susceptibility to breast cancer has been linked to mutations of the BRCA-1 and BRCA-2 genes. Proteins encoded by these genes are believed to work to preserve chromosome structure, but their precise role is unclear due to them being involved in a multitude of processes. It is postulated that a mutation causes a disruption in the protein which causes chromosomal instability in BRCA deficient cells thereby predisposing them to neoplastic transformation.

About 10% of breast cancer cases cluster in families, some due to mutations in the BRCA-1 and BRCA-2 genes, giving rise to higher cancer risk. Mutations in other genes linked to tumor suppression may account for cancer predisposition. These include mutations in p53 tumor suppression, the STK11/LKB, protein kinase or the PTEN phosphatase.

Apart from the existence of mutations in tumor suppressor genes, there is no dear picture of how these genes interact which manifests in a predisposition or development of tumors. BRCA1 associated cancers have a unique histopathology and demonstrate more frequent loss of expression of the tumour suppressor protein p27^{Kip1} than in sporadic cases. p27^{Kip1} normally plays a role in maintaining G1 arrest inhibiting cyclin-CDK complexes and is degraded upon mitogen stimulation to re-enter the cell cycle. p27^{Kip1} has an accepted role as a tumour suppressor gene and poor prognosis associates with loss of expression in a wide array of cancers.

Low p27 levels are commonly seen in many human cancers such as epithelial cancers including, lung cancer, gastrointestinal malignancies, breast cancer, prostate cancer, ovarian cancer, and carcinomas of the larynx and oral cavity. The correlation is not as clear for brain tumors and malignant lymphomas.

p27, as a prognostic tool has been used for breast cancer. This is the most common solid tumor and one of the most frequent causes of death in women. It has been shown that both low levels of p27 and high levels of cyclin E were independent predictors of overall survival. Patients whose breast cancers showed both low p27 and elevated cyclin E proteins had the highest mortality. An inverse correlation between cyclin E levels and p27 has also been observed in breast cancers, whereas increased levels of cyclin D1 were associated with both higher p27 levels and low tumor grade (a greater degree of differentiation) in another study. Accordingly a correlation of p27 and that of cycline is not always a clear and absolute indication of the predisposition for the cancer.

A low level of p27 may influence many cell functions. Because it is actively involved in various stages of the cell cycle and its role in cell progression is complex it may also be associated with apoptosis, differentiation and responses to cellular stresses, such as, but not limited to, DNA damage.

The relevance of p27 to tumor phenotypes provides some guidance as to pathways regulating its interaction with other molecules within the cell. A direct correlation of the p27 levels to the cancer has not been made and it is unclear whether such a correlation does exist particularly when p27 is involved in many stages of the cell cycle. The action of p27 is not clearly understood. Its importance however may lead to the generation of new and effective drugs for cancer. A specific target which can be used to produce the effective drug remains elusive.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed in Australia before the priority date of each claim of this application.

Accordingly, it is an object of the present invention to identify a suitable target of a BRCA associated disorders, especially a BRCA-1 associated disorder such as a cancer, more preferably a breast cancer which may be used to develop useful and effective drugs for cancer treatment.

### SUMMARY OF THE INVENTION

In a first aspect of the present invention there is provided a method of treating a BRCA associated disorder, said method comprising modulating interaction between a BRCA gene or gene product and a p27 gene or gene product.

Applicants have found for the first time that the loss of p27 protein is functionally important in the progression of BRCA associated cancers. Whilst p27 levels have been found to be low in various cancers, evidence of a causal link between the two has not previously been shown to have an effect in establishing or reversing the phenotype.

In a preferred embodiment the method involves modulating interaction between a BCRA gene or gene product and a p27 gene or gene product by modulating expression and/or activity of a cdk2 protein, preferably reducing the expression and/or activity of the cdk2 protein.

Applicants have found that by specific targeting of cdk2 and reducing the activity of this kinase, the BRCA associated disorder may be treated. It is postulated that p27 would inhibit cdk2. Low levels of p27 in cancer fails to inhibit cdk2 and hence cell cycling and proliferation in cancers is not inhibited.

In a further preferred aspect of the invention, there is provided a method of treating a BRCA associated disorder in a patient, said method comprising administering a therapeutically effective amount of a cdk-2 inhibitor or antagonist to the patient.

In yet another aspect of the present invention, there is provided a transgenic animal model for breast cancer wherein said animal shows a clinical interaction between BRCA and p27 said animal comprising

a promoter-driven dominant negative BRCA allele.

In yet another aspect of the present invention, there is provided a method of screening potential therapeutics for treatment of BRCA associated disorder, said method comprising:

inducing a BRCA associated disorder phenotype in a transgenic animal model for the BRCA associated disorder, said animal comprising

a promoter driven dominant negative BRCA allele;

exposing a potential therapeutic to the transgenic animal; and

noting a change in the phenotype.

### FIGURES

FIG. 1 shows a reduction in the dosage of p27^{kip1} alters the phenotype in MMTV-trBRCA-1 expressing mice.

FIG. 2 shows MMTV-trBRCA-1 and reduced p27 dosage interact during both alveolar development and ductal branching.

FIG. 3 shows that a reduced dosage of p27 does not reduce the expression of the MMTV-trBRCA-1 transgene.

FIG. 4 shows staining for p27^{kip1} corresponds to regions with low proliferation.

FIG. 5 shows an analysis of primary mammary epithelia cells (PMECS) from transgenic mice.

### DESCRIPTION OF THE INVENTION

In a first aspect of the present invention there is provided a method of treating a BRCA associated disorder, said method comprising modulating interaction between a BRCA gene or gene product and a p27 gene or gene product.

Applicants have found for the first time that the loss of p27 protein is functionally important in the progression of BRCA associated caners. Whilst p27 levels have been found to be low in various cancers, evidence of a causal link between the two has not previously been shown to have an effect in establishing or reversing the phenotype.

As used herein, a “BRCA associated disorder” is a disorder associated with a BRCA protein, preferably a BRCA-1 or related proteins including BRCA-1 and BARD1; or BRCA-2 protein encoded by a mutated BRCA gene. Preferably the disorder is associated with a mutated BRCA-1 or BRCA-2 gene. The disorder may manifest as a cancer, preferably an epithelial cancer including but not limited to lung cancer, gastrointestinal malignancies, breast cancer, prostate cancer, ovarian cancer and carcinomas of the larynx and oral cavity. More preferably the cancer is a breast or ovarian cancer. Most preferably, it is a breast cancer.

“Treatment” as used herein includes in its broadest sense to include prophylactic (ie preventative) treatment as well as treatments designed to ameliorate the effects of the disorder. The treatment may be aimed at the disorder or at the effects of the disorder. Additionally, treatment may include reversing phenotypes characteristic of the disorder.

A cell may show a predisposition to a BRCA associated disorder, by identification of prognostic markers, including but no limited to, oncogenes or genes which are indicative of the cancer such as mutations of the BRCA gene including mutations of the BRCA-1 or BRCA-2 tumor suppression genes.

Preferably, the BRCA associated disorder coincides with low levels of p27. Cancers showing low levels of p27 include the epithelial cancers described above. Preferably, the cells are BRCA1 or BRCA2 negative or include mutated BRCA-1 or related proteins including BAP-1 or BARD-1 or mutated BRCA-2 protein. Preferably the cells of the cancers show mutated BRCA genes, more preferably mutated BRCA-1 or BRCA-2 genes indicating, a predisposition for breast cancer.

It is preferred that to treat the BRCA associated disorder, an increased interaction between the BRCA gene or gene product with a p27 gene or gene product is induced. These may be achieved by increasing the p27 protein which is low in BRCA associated disorders.

Applicants have found that loss of p27 protein is functionally important in the progression of BRCA-1 associated cancers. Hence, increased p27 protein may reverse the effects of BRCA-1 mutations and a predisposition to cancer.

As used herein “modulating interaction” is meant to describe a change in interaction between at least two integers. “Modulating” may include direct or indirect modulation. Direct modulation acts directly on the integers involved whilst indirect modulation acts indirectly by targeting downstream or upstream parameters that may affect the integrity of the integer thereby changing the manner in which they may interact.

The “BRCA gene and gene products” as referred herein is preferably a BRCA-1 or BRCA-2 gene or gene product.

The “p27 gene or gene products” as referred herein is preferably the p27^{kip1} gene or gene product.

The method of treatment may be performed by introducing a compound which modulates an interaction between BCRA gene and gene products and p27 gene and gene products to a site of the BRCA associated disorder. For instance, if the disorder is a cancer, such modulators 6nay be directly injected into the tumor or if the cancer is more wide spreads the compound my be introduced by injection either subcutaneously, intravenously, intraperitoneally, intradermally, intramuscularly, mucosally or any combination of these. The compound may be injected in soluble form, aggregate form, attached to a physical carrier or mixed with an adjuvant, using methods and materials known in the art.

Throughout the description and claims of this specification, the word “comprise” and variations of the word, such as “comprising” and “comprises”, is not intended to exclude other additives, components, integers or steps.

In a preferred embodiment the method involves modulating interaction between a BCRA gene or gene product and a p27 gene or gene product by modulating expression and/or activity of a cdk2 protein, preferably reducing the expression and/or activity of the cdk2 protein.

Cell cycle progression is governed by cyclin dependent kinases (cdks) that are activated by cyclin binding and inhibited by the cdk inhibitors. The cdks regulate biochemical pathways, or check-points, that integrate mitogenic and growth-inhibitory signals and coordinate cell-cycle transitions. Passage through G1 into S phase is regulated by the activities of cyclin D-, cyclin E-, and cyclin A-associated cdks. B-type cyclin associated kinases regulate the G2/M transition.

Members of the kinase inhibitor protein (kip) family, p21^{Kip1}, p27^{Kip1}, and p57^{Kip2}, bind and inhibit cyclin-bound cdks. Although p21 and p27 are inhibitors of cyclin E/cdk2, they may also function in the assembly and activation of cyclin D-cdk4 and -cdk6 complexes.

The cdk inhibitor p27 acts in G0 and early G1 to inhibit G1 cyclin cdks, with the target generally being E-type cyclin/cdk2 complexes. Mitogenic growth factor signalling causes loss of p27, and p27 levels and/or activity increase in response to different signals. p27 mRNA levels are constant throughout the cell cycle and p27 protein levels are regulated by translational controls by ubiquitin-mediated proteolysis.

Without being limited by theory, this method utilises the discovery that the activity of cdk2 is important in maintaining the phenotype and progression of the BRCA associated disorder, preferably in cancer, most preferably in breast cancer. By reducing the activity of cdk2 or inhibiting cdk2, selective toxicity for cancers, particularly breast cancer, more particularly a BRCA1 mutant breast cancer can be applied to reduce cell cycling and progression of the phenotype.

The protein cdk2 is closely involved in cell cycle progression. However, it is not the only cyclin dependent kinase involved. Other cyclin dependent kinases include cdk4 and cdk6 activity involved in specific stages of the cell cycle. In particular these cyclin dependent kinases are involved in the G1 phase whereas cdk2 can be involved in the G0/G1/S phase. The activity of these kinases may be regulated by binding of the cyclins, by phosphorylation and by negative regulators or cdk inhibitors such as p15, p16, p18, p19, p21, p27 and p57.

Applicants have found that by specific targeting of cdk2 and reducing the activity of this kinase, the BRCA associated disorder may be treated. It is postulated that p27 would inhibit cdk2. Low levels of p27 in cancer fails to inhibit cdk2 and hence cell cycling and proliferation in cancers is not inhibited.

“Activity” as used herein relates to a function of the cdk2 protein, and includes the ability of cdk2 to stimulate proliferation or growth by binding to chaperone, or upstream or downstream effector molecules thereby activating or repressing upstream or downstream pathways which affect proliferation or differentiation.

The term “modulating expression and/or activity” as used herein includes modifying or altering the expression and/or activity of cdk2 protein, compared to unmodified levels of cdk2. Expression and/or activity may be increased or decreased compared to unmodified levels to increase or decrease proliferation or differentiation.

Modulation of cdk2 expression and/or activity in the cell may be achieved using antagonists, inhibitors, mimetics or derivatives of the cdk2 in a direct fashion. The terms “antagonist” or “inhibitor”, as used herein, refer to a molecule which, when bound to cdk2, blocks, inhibits or modulate the activity of cdk2. Antagonists and inhibitors may include proteins, nucleic acids, carbohydrates, antibodies or any other molecules including ligands which bind to cdk2. Proteins may include enzymes which can degrade cdk2 and therefore affect the level of cdk2 exposed to the cell. Other modulators of the activity and/or expression of cdk2 include a range of rationally-designed, synthetic inhibitors.

Modulation of cdk2 expression and/or activity may also be achieved by indirect methods. Apart from the direct methods describe above, modulation of expression and/or activity of cdk2 may be achieved using direct recombinant methods known to those of skill in the art and include, but are not limited to, knockout technology, antisense technology, triple helix technology, targeted mutation, gene therapy, regulation by agents acting on transcription. Indirect methods for modulating expression and/or activity of cdk2 may include targeting upstream or downstream regulators of cdk-2. More specifically, cdk-inhibitors such as p15, p16, p18, p19, p21, p27 and p57 may b used. Other modulators include other cdk's including cdk4, cdk-6, cdk-7 or cdc25A-C, Wee1-family kinases including Myt1, RB-family proteins pRB, p107 and p130.

Activity of cdk2 can be measured by methods available to the skilled addressee including phosphorylatlon of substrates such as pRB or indirectly by determining cell proliferation.

In a further preferred aspect of the invention, there is provided a method of treating a BRCA associated disorder in a patient, said method comprising administering a therapeutically effective amount of a cdk-2 inhibitor or antagonist to the patient.

Preferably the BRCA associated disorder is a BRCA-1 or BRCA-2 disorder and may be any one of the disorders described above. Preferably, the disorder is a BRCA-1 disorder, more preferably, it is a breast cancer

The cdk-2 inhibitor or antagonist may be administered in any manner which delivers the inhibitor or antagonist to the site to be treated. Carriers which target the cancer cells may be coupled to the inhibitors or antagonists to effectively deliver the compound to the cells of interest.

A therapeutically effective dose as used herein refers to that amount of a compound sufficient to result in a healthful benefit in the treated subject.

Toxicity and therapeutic efficacy may be determined by standard pharmaceutical procedures that may involve cell cultures or experimental animals, e.g., for determining the LD₅₀ (the dose lethal to 50% of the population) and the ED₅₀ (the dose therapeutically effective in 50% of the population). The data obtained from the cell culture assays and animal studies can be used in formulating a range of dosage for use in humans. The dosage of a compound lies preferably within a range of circulating concentrations that include the ED₅₀ with little or no toxicity. The dosage can vary within this range depending upon the dosage form employed and the route of administration used in the method of the invention.

The inhibitors or antagonists may be formulated into compositions. The compositions can be administered to humans and other animals orally, rectally, parenterally (i.e. intravenously, intramuscularly, or sub-cutaneously), intracisternally, intravaginally, intraperitoneally, topically (as by powders, ointments, or drops), transdermally, bucally, or as an oral or nasal spray.

The compositions for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), and suitable mixtures thereof, vegetable oils (such as live oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of microorganisms may be ensured by the inclusion of various antibacterial and antifungal agents, for example, paraben, chlorobutanol phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

If desired, and for more effective distribution, the compounds can be incorporated into slow release or targeted delivery systems such as polymer matrices, liposomes, and microspheres.

The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

If desired, and for more effective distribution, the compounds can be incorporated into slow release or targeted delivery systems such as polymer matrices, liposomes, and microspheres.

The active compounds can also be in microencapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Dosage forms for topical administration of a compound of this invention include powders, sprays, ointments and inhalants. The active compound is mixed under sterile conditions with a pharmaceutically acceptable carrier and any needed preservatives, buffers, or propellants which may be required.

One skilled in the pertinent art will recognize that suitable dosages for any of the modulations described herein will vary, depending upon such factors as the nature of the disorder to be treated, the patient's body weight, age, and general condition, and the route of administration. Preliminary doses can be determined according to animal tests, and the scaling of dosages for human administration is performed according to art-accepted practices.

In yet another aspect of the present invention, there is provided a transgenic animal model for breast cancer wherein said animal shows a clinical interaction between BRCA and p27 said animal comprising

a promoter-driven dominant negative BRCA allele.

The transgenic animal provides a model which replicates an interaction between BRCA and p27. Preferably the model includes a null allele of p27.

The BRCA may be either a BRCA-1 or BRCA-2. Preferably, the BRCA is BRCA-1 and more preferably, the animal expresses a truncated BRCA-1, more preferably comprising amino acids 1-299.

The BRCA may be promoter driven and any promoter which can induce the expression of BRCA may be used. Preferably, the promoter is an MMTV promoter.

The animals ideally carry null alleles of p27, preferably p27^{Kip1}. This is particularly useful for replicating a loss of p27 which has been shown to be functionally important to the progression of BRCA-1 associated cancers.

The transgenic animal may be any animal, however, mice are particularly preferred.

In a further preferred aspect, the transgenic animal contains a p27 allele which expresses p27, preferably under the control of a promoter. The animal may be induced via the BRCA-1 allele to show phenotypic characteristics of breast cancer. A promoter driven p27 may be used to reverse phenotypic characteristics of the BRCA-1 allele.

In yet another aspect of the present invention, there is provided a method of screening potential therapeutics for treatment of BRCA associated disorder, said method comprising:

Inducing a BRCA associated disorder phenotype in a transgenic animal model for the BRCA associated disorder, said animal comprising a promoter driven dominant negative BRCA allele;

exposing a potential therapeutic to the transgenic animal; and

noting a change in the phenotype.

This method utilizes a transgenic mouse model which substantially reproduces an induction of cancer. In particular, the BRCA associated disorder is a BRCA-1 gene associated disorder, preferably breast cancer. The breast cancer may arise due to the interaction of BRCA-1 and low p27 levels. Preferably, the transgenic animal carries a null allele of p27 and the BRCA-1 gene can be induced by a suitable promoter such as MMTV. The combination of BRCA-1 and low levels of p27 induces conditions of breast cancer.

The potential therapeutic may be administered to the animal by any route, including but limited to oral, rectal, parenteral, intracisternal, intravaginal, intraperitoneal, topically, transdermal or as an oral or nasal spray.

Preferably, the potential therapeutic is a compound which modulates expression and/or activity of cdk2 protein. Preferably, the compound inhibits expression and/or activity of cdk2 protein.

The change in phenotype may be noted physically or by changes in the phenotype or of the change in progression of the breast cancer phenotype. This may be noted a number of ways including cell proliferation and growth, change a tissue cross section or p27 levels. Preferably, the phenotype is reversed, more preferably p27 levels revert closer to normal levels in the absence of breast cancer. These changes can be compared with animals not exposed to the potential therapeutic.

Examples of the procedures used in the present invention will now be more full described. It should be understood, however, that the following description is illustrative only and should not be taken in any way as a restriction on the generality of the invention described above.

### EXAMPLES

### Example 1

### Characterisation of a Genetic Interaction Between brca1 and p27^{kip1} in the Mouse Mammary Gland

Breast cancers arising in individuals carrying germline mutations in BRCA1 often display pathological characteristics and a spectrum of secondary genetic mutations that differs from the majority of sporadically derived breast cancers. Several clinical reports show an increased frequency of low expression of the CDK-inhibitor p27^{Kip1} in Brca1-associated cancers, when compared to sporadic cancers.

Mice expressing an MMTV driven C-terminal truncating allele of Brca1 (MMTV-trBrca1) that is putatively dominant negative over the wild type protein have been generated. Mice expressing MMTV-truncated Brca1 (aa 1-299) were generated at the ICRF transgenic facility. Mice carrying null alleles of p27^{Kip1} were kindly provided by Jim Roberts laboratory (Fero et al, Cell. May 31, 1996; 85(5):73344.). Mice are backcrossed at least five generations to the FVB/N strain.

Such mice display blunted ductal development of the mammary gland, very similar to the phenotype seen in mice with a mammary specific conditional knockout of Brca1. To investigate the cellular and molecular basis of a genetic interaction between p27^{Kip1} and Brca1, mice expressing the trBrca1 transgene on the background of null alleles of the p27^{kip1} gene ware generated.

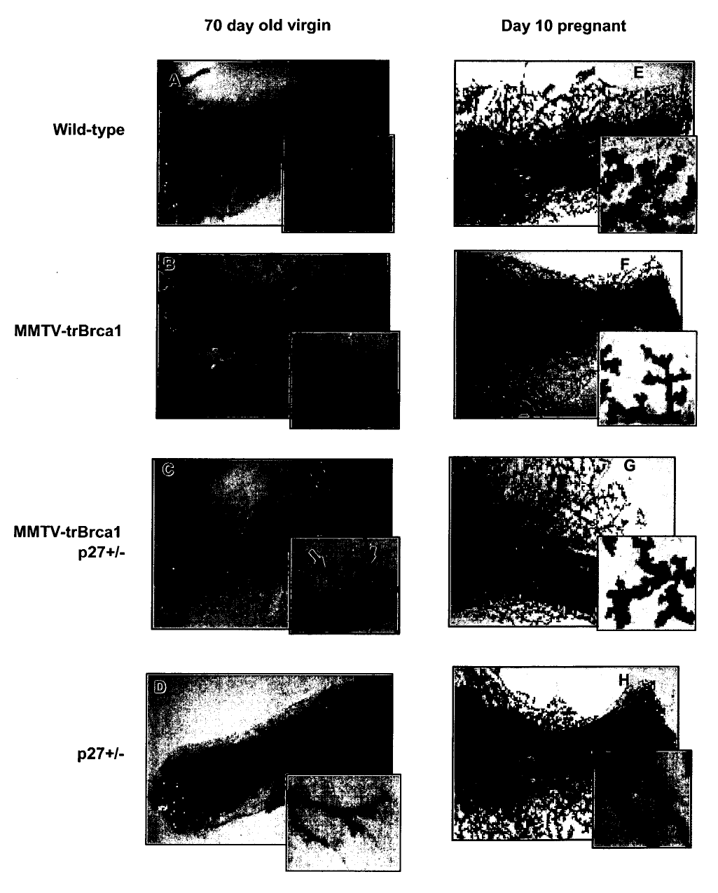

To conduct histochemistry, mice were injected with 100 μg BrdU/gram of body weight 1 hr prior to sacrifice. Mammary glands were harvested, whole mounted, fixed in Camoy's fixative and stained with carmine red or fixed in 10% buffered formalin and embedded in paraffin. Immunohistochemistry was performed on 4μ sections using anti-BrdU (BD) or anti-p27 (DAKO). FIG. 1 shows reduction in the dosage of p27Kip1 alters the phenotype in MMTV-trBrca1 expressing mice. Wholemounts of mammary tissue from MMTV-trBrca1 mice gland (B,F) show blunted ductal morphogenesis as virgins and during pregnancy compared to wildtype (A,E) at the same stage (red vs black arrows). p27 deficient mice carrying the transgene (C,G) do not show blunted ductal development (blue vs red arrows), while p27 deficiency alone does not convey a phenotype (D,H). This interaction is also observed in 42 day old virgins and at day 16.5 of pregnancy (not shown) while approximately 50% of MMTV-trBrca1 p27± virgin mice show precocious alveolar development (C, blue arrows). Wholemounts are stained with carmine red.

Characterisation of mammary development in MMTV-trBrca1 p27^{Kip1}-deficient mice are provided.

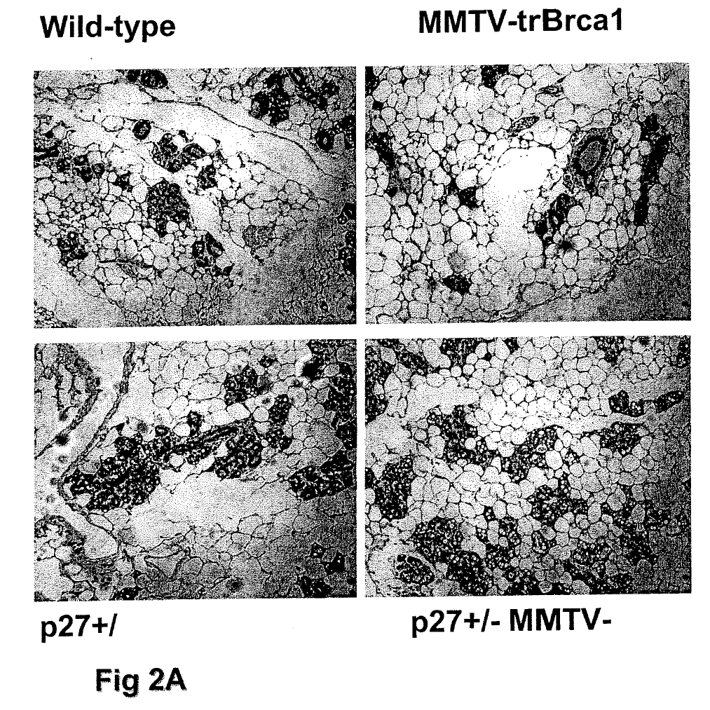

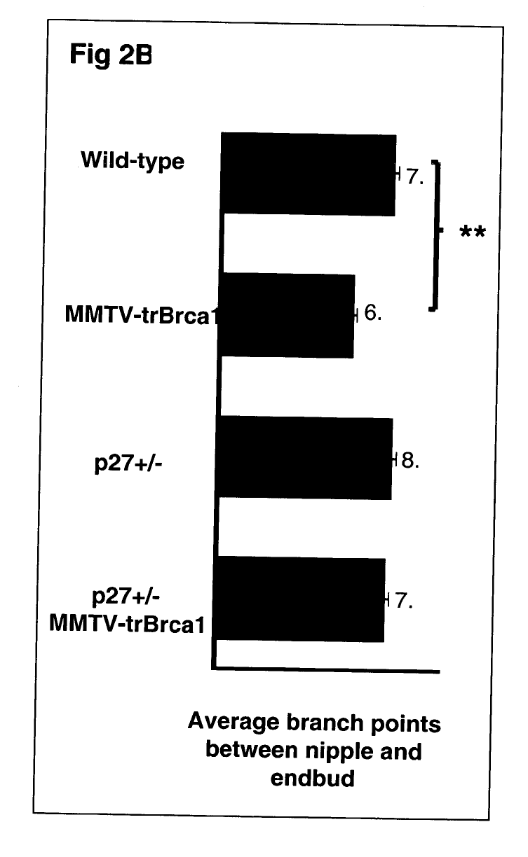

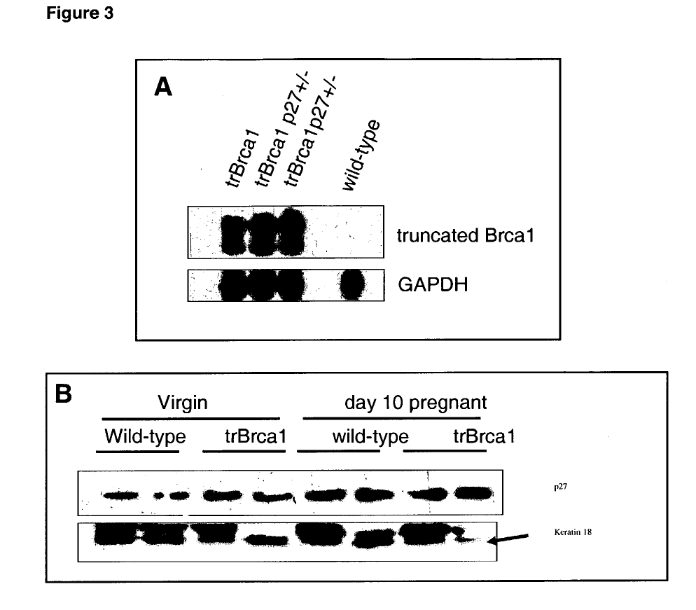

FIG. 2 shows MMTV-trBrca1 and reduced p27 dosage interact during both alveolar development and ductal branching. A. Haemotoxylin and eosin staining of day 10 pregnant mammary sections demonstrates the failure of alveolar development in MMTV-trBrca1 compared to wild-type, p27± and p27± trBrca1 mice. B. Scoring of the number of branch points between nipple and endbud in 70 day old virgin mice demonstrates reduced ductal branching in MMTV-trBrca1 mice vs wild-type (**p>0.01). Reduced p27 dosage rescues the branching to wild-type levels. Snap frozen mammary tissue was thawed and lysed in cyclin E kinase buffer (protein) or Trizol (RNA) using a power homogeniser. Protein was quantitated using a BCA detection method. RNA was quantitated at A₂₆₀ FIG. 3 shows A Reduced dosage of p27 does not reduce the expression of the MMTV-trBrca1 transgene as shown by Northern blot. RNA was probed with the 5′ 1 kb EcoR1 fragment of the Brca1 cDNA and a Pst1 fragment of GAPDH as loading control. B p27 protein levels are slightly elevated in whole mammary extracts in virgins and at day 10 pregnancy. Results are confounded by disproportionate epithelial content in whole extracts as demonstrated by keratin 18 staining.

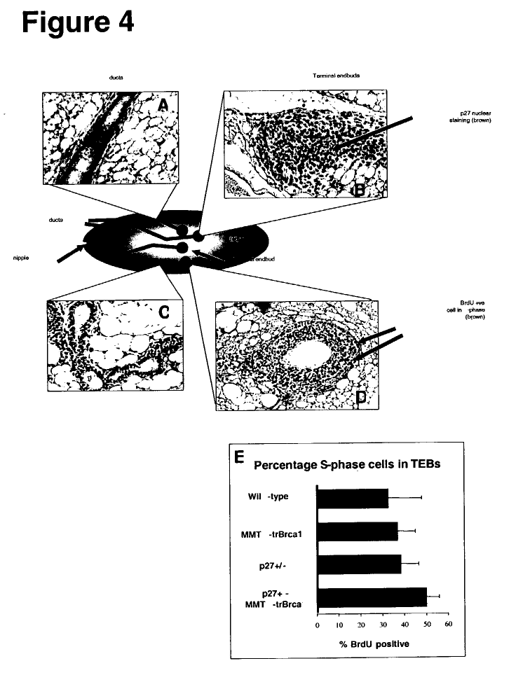

FIG. 4 shows staining for p27^{Kip1} corresponds to regions with low proliferation, measured by BrdU incorporation. In the ducts, all nuclei are p27 positive (A) while there are <2% BrdU positive cells (C). In the terminal end-buds (TEBs) of 42 day old mice, less than half of the cells demonstrate measurable p27 (B) while S-phase is close to 40% (D) in this rapidly proliferating region.

No significant difference in BrdU incorporation is seen between wild-type, MMTV-trBrca1 and p27± TEBs, although a trend towards higher S-phase content is seen in p27± trBrca1 TEBs (E). Results are average % BrdU positivity (±SD) of at least 5 TEBs from 3 mice, with TEBs identified according to morphological characteristics. Aseptically harvested mammary tissue was finely minced and digested in collagenase. Primary Mammary Epithelial Cells (PMECs) were isolated by differential centrifugation and cultured in defined medium on collagen coated plates. FACs, protein and RNA isolation were performed on cells at passage 2.

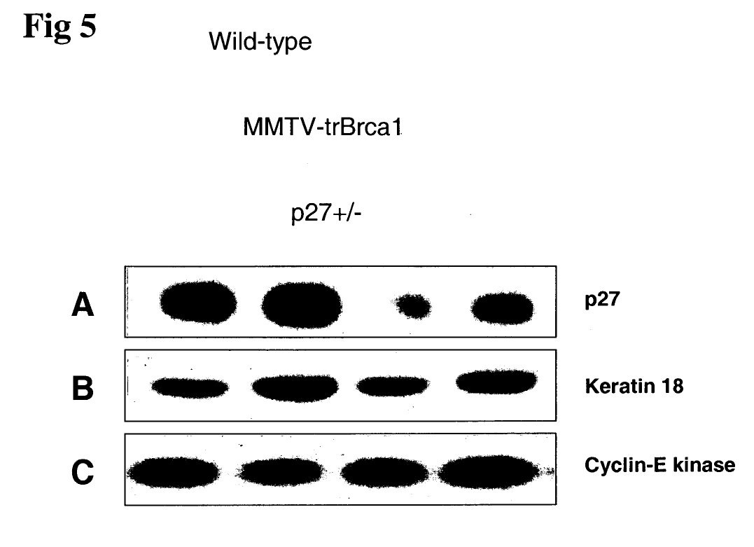

FIG. 5 shows analysis of primary mammary epithelial cells (PMECs) from transgenic mice. p27^{Kip1} protein levels are less than 50% wild-type levels in p27± mice, while there is a trend towards higher expression in mice with the MMTV-trBrca1 transgene. p21^{Cip1}, cyclin D1 and cyclin E show no significant changes.

These results may provide insight into why BRCA1 associated cancers consistently demonstrate low levels of p27^{Kip1}.

These studies show:

Reduction in p27^{Kip1} gene dosage reverses the blunted ductal and alveolar development seen in mice expressing the dominant negative MMTV-trBrca1 gene. We believe that loss of Brca1 function initially confers a cellular disadvantage and that this is overcome on a p27-deficient background.

Our model supports the notion that loss of p27 protein is functionally important to the progression of Brca1-associated cancers although conditions used to culture primary mammary epithelial cells in the short term are not sufficient to replicate the in vivo results.

The mechanism of the in vivo interaction is yet to be determined, although we hypothesise that it is due to rescue of an apoptotic phenotype requiring p27^{Kip1}.

Finally it is to be understood that various other modifications and/or alterations may be made without departing from the spirit of the present invention as outlined herein.

1. A method of treating a BRCA associated disorder, said method comprising modulating interaction between a BRCA gene or gene product and a p27 gene or gene product.

2. A method according to claim 1 wherein the BRCA associated disorder is associated with a disorder of a BRCA-1 or BRCA-2 gene or gene product.

3. A method according to claim 1 wherein the disorder results from a mutation of the BRCA-1 or BRCA-2 gene.

4. A method according to claim 2 wherein the gene product is selected from the group including a BRCA-1 protein, BAP-1 or BARD1.

5. A method according to claim 1 wherein the BRCA associated disorder is selected from the group including lung cancer, gastrointestinal malignancies, breast cancer, prostate cancer, ovarian cancer and carcinomas of the larynx and oral cavity.

6. A method according to claim 5 wherein the disorder is a breast cancer.

7. A method according to claim 1 wherein the interaction between the BRCA gene or gene product and the p27 gene or gene, product is modulated by increasing p27 protein.

8. A method according to claim 1 wherein the p27 gene or gene product is a p27^{kip1} gene or gene product.

9. A method according to claim 1 wherein the interaction between the BRCA gene or gene product and the p27 gene or gene product is modulated by modulating expression and/or activity of a cdk-2 protein.

10. A method according to claim 9 wherein the expression and/or activity of the cdk-2 protein is reduced.

11. A method according to claim 10 wherein the expression and/or activity of the cdk2 protein is reduced by a cdk inhibitor or modulator selected from the group including p15, p16, p18, p19, p21, p27, p57, cdk-4, cdk-6, cdk-7, cdc25A-C, Wee1-family kinases including Myt1, RB-family proteins pRB, p107 and p130.

12. A method of treating a BRCA associated disorder in a patient, said method comprising administering a therapeutically effective amount of a cdk-2 inhibitor or antagonist to the patient.

13. A method according to claim 12 wherein the BRCA associated disorder is associated with a disorder of a BRCA-1 or BRCA-2 gene or gene product.

14. A method according to claim 13 wherein the disorder results from a mutation of the BRCA-1 or BRCA-2 gene.

15. A method according to claim 12 wherein the BRCA associated disorder is selected from the group including lung cancer, gastrointestinal malignancies, breast cancer, prostate cancer, ovarian cancer and carcinomas of the larynx and oral cavity.

16. A method according to claim 15 wherein the disorder is breast cancer.

17. A method according to claim 12 wherein the cdk-2 inhibitor or antagonist is selected from the group including p15, p16, p18, p19, p21, p27, p57, cdk-4, cdk-6, cdk-7, cdc25A-C, Wee1-family kinases including Myt1, RB-family proteins pRB, p107 and p130.

18. A transgenic animal model for breast cancer wherein said animal shows a clinical interaction between BRCA and p27 said animal comprising
a promoter-driven dominant negative BRCA allele.

19. A transgenic animal according to claim 18 wherein the allele is a null allele of p27.

20. A transgenic animal according to claim 19 wherein the null allele of p27 is a p27^{kip1}.

21. A transgenic animal according to claim 18 which expresses a truncated BRCA-1 comprising amino acids 1 to 299 or BRCA-1.

22. A method of screening potential therapeutics for treatment of a BRCA associated disorder, said method comprising:
inducing a BRCA associated disorder phenotype in a transgenic animal model for the BRCA associated disorder, said animal comprising a promoter driven dominant negative BRCA allele;
exposing a potential therapeutic to the transgenic animal; and
noting a change in the phenotype.

23. A method according to claim 22 wherein the BRCA associated disorder is a BRCA-1 gene associated disorder.

24. A method according to claim 23 wherein the disorder is breast cancer.

25. A method according to claim 22 wherein the disorder is induced by modulating interaction between a BRCA gene or gene product and a p27 gene or gene product.

26. A method according to claim 25 wherein induction results form an interaction between BRCA-1 and low levels of p27.

27. A method according to claim 22 wherein the potential therapeutic is a compound which modulates expression and/or activity of cdk-2 protein.
